(19) United States
(12) Patent Application Publication
(10) Pub. No.: US 2012/0071409 A1
(43) Pub. Date: Mar. 22, 2012

(54) Colloidal Liposomal Compositions and Methods

(76) Inventors: Jacob C. Hack, (US); Richard D. Tobias, (US); Kenneth Blum, San Diego, CA (US); B. William Downs, Laderach, PA (US); Roger L. Waite, San Diego, CA (US); William J. Heaney, Huntington Beach, CA (US); John J. Giordano, Miami, FL (US)

(21) Appl. No.: 13/043,442

(22) Filed: Mar. 8, 2011

Related U.S. Application Data
(63) Continuation-in-part of application No. 12/105,266, filed on Apr. 18, 2008, now abandoned.
(60) Provisional application No. 60/912,465, filed on Apr. 18, 2007.

(51) Int. Cl. A61K 38/27; A61K 47/44

(52) U.S. Cl. 514/11.3; 514/785

(57) ABSTRACT

A formulation delivery system is disclosed. When configured as a pro-liposome oral tablet, the tablet comprises a blend of at least one Iiposome forming ingredient, at least one active ingredient and at least one co-disintegrant. After ingestion at least a portion of the tablet disintegrates in a portion of an alimentary system to provide one or more in situ Iiposome encapsulated active ingredients. A method of producing the pro-liposome oral tablet and a method of therapeutically dosing with the pro-liposome oral tablet are further disclosed. The formulation delivery system may be configured as any one of a pro-liposome oral tablet, sublingual tablet, caplet, capsule and aerosol.

### CROSS-REFERENCE TO RELATED APPLICATIONS

This application claims priority to and the benefit of U.S. Provisional Application No. 60/912,465, filed Apr. 8, 2007, entitled “Oral Tablet Dosage Forms”, and U.S. patent application Ser. No. 12/105,266, filed Apr. 18, 2008, entitled “Dosage Forms and Methods Thereof”, of which this application is a continuation-in-part. Where permitted, the foregoing applications are incorporated by reference, each in its entirety, for any and all purposes.

### FIELD OF THE DISCLOSURE

This disclosure relates generally to oral tablets and, more specifically, to improved oral tablets or other effective iiposome compositions configured with one or more dosage forms to be encapsulated by one or more Iiposome derivatives to be introduced in a specific portion of a bodily system and improved methods for providing therapeutically effective amounts of in situ Iiposome active ingredients.

### BACKGROUND OF THE DISCLOSURE

The technology of producing liposomes is fairly mature. Indeed, various nutritional supplements have been formulated in conjunction with liposomes to provide for improved ways of delivering effective doses of the nutritional supplements. Many nutritional supplements may be degraded when taken orally so that their delivery may not be therapeutically effective.encapsulating nutritional supplements with one or more phospholipids to form lipsomes provides a measure of protection for the nutritional supplements and may enhance their therapeutic effect. Of course, other capsule and tablet forms of protecting nutritional supplements exist, but when the capsules or tablets disintegrate in the alimentary system, the nutritional supplements may not be as well received and delivered to target organs as are encapsulated lipsome nutritional supplements combinations that may be in liquid or aerosol forms too depending on a best desired method of introducing such pharmaceutically active ingredients. Further, as is understood in the art, such active pharmaceutical ingredients may further enter the bodily system by passing through skin, and thus enter the blood stream, where in the course of time, targeted organs may benefit from yet another way of introducing active pharmaceutical ingredients. As is understood in the art skin is generally considered to be fairly impermeable to water. However, under appropriate conditions, as is understood in the art, some pharmaceutically active ingredients actually are able to penetrate the skin and enter into muscles tissues and the blood stream to relieve conditions such as arthritis and even muscle soreness due to overuse of such tissue. Appropriate exercise and appropriate foods and beverages have also been considered to be important components of maintaining good health in immune compromised individuals. Indeed, even when an individual's health is not poor, their immune system may be under attack at all times. Thus a healthy life style plus appropriate Iiposome nutritional supplements combinations comprising pharmaceutically active ingredients may be highly desirable to continue to remain in good overall health.

It has proved to be difficult to provide stable forms of Iiposome encapsulated nutritional supplements because the technology of making stable liposomes with long term stability is experimentally challenging. Often, the encapsulated nutritional supplements fall out of the Iiposome components and therefore are no longer encapsulated. Storage conditions must be rigorously controlled. Additionally, attempts to improve the stability of such Iiposome encapsulated nutritional supplement combinations have often proved to be specific to the types of nutritional supplements to be delivered and requires considerable formulating expertise.

Consequently, a need exists for improved products and methods for delivering an effective quantity of one or more nutritional supplements (or pharmaceutically active ingredients), wherein the one or more nutritional supplements reach their targeted location prior to any significant degradation. Of course, any appropriate Iiposome delivery pharmacokinetic method may also provide improved products and methods for targeting appropriate locations of the immune system too.

### SUMMARY OF THE DISCLOSURE

The current disclosure provides one or more oral (or sublingual) tablets comprising one or more phospholipids and derivatives thereof and one or more dosage forms (or active pharmaceutical ingredients), wherein at least a portion of the active pharmaceutical active ingredients may be encapsulated and enter into a person's alimentary system to provide one or more encapsulated liposomes. Such oral tablets may be more bio-available because of being encapsulated in situ as desired rather than being pre-encapsulated. Furthermore, the disclosed combinations are substantially non-irritant when consumed and digested. A further advantage is that self-dosage with the disclosed formulations is simple to understand and apply.

In accordance with an embodiment of this disclosure, a pro Iiposome oral tablet is disclosed. In an exemplary aspect, the oral tablet may comprise in combination, an oral tablet comprising about 77% excipients, about 17% co-disintegrants, about 1% of one or more active ingredients and about 5% of phospholipids and phospholipid derivates thereof. The oral tablet may further comprise at least one preservative. Furthermore, the oral tablet may comprise at least one prodrug.

In an embodiment the oral tablet may comprise one or more phospholipids and mixtures thereof including the following: phosphatidyl ethanolamine, distearylphosphatidyl choline, dimyristylphosphatidyl choline, phosphatidyl choline, dipalmitoylphosphatidyl glycerol, dipalmitoylphosphatidyl choline, dipalmitoylphosphatidic acid and lecithin.

Furthermore, the oral tablet may comprise one or more co-disintegrants and mixtures thereof including the following: starch, crosscarmelose, crospovidone, sodium starch glycolate, micro crystalline cellulose, lactose, dextrose, gelatin, gum acacia, stearic acid, calcium phosphate, alginic acid, calcium carboxymethylcellulose, sodium carboxymethylcellulose, methylcellulose and other carboxymethylcellulose salts thereof, hydroxyethylcellulose, hydroxypropylcellulose, hydroxypropylmethylcellulose phthalate, non-crystalline cellulose, polyvinyl alcohol, and polyvinyl pyrrolidone.

In an embodiment the dosage form (or active ingredients) may comprise one or more active homeopathic ingredients. In an exemplary embodiment an active homeopathic ingredient may comprise one or more somatotropins. In a further embodiment the dosage form may comprise an allopathic ingredient.

In accordance with another embodiment of this disclosure, a method of producing a pro Iiposome oral tablet comprising about 77% excipients, about 17% co-disintegrants, about 1% of one or more dosage forms and about 5% of phospholipids and derivates thereof is disclosed.

Furthermore, in yet another embodiment of this disclosure a method of dosing a person with a pro Iiposome oral tablet comprising about 77% excipients, about 17% co-disintegrants, about 1% of one or more dosage forms and about 5% of phospholipids and derivates is disclosed.

In accordance with further embodiments of this disclosure pro Iiposome capsules and pro Iiposome aerosols have been disclosed.

The foregoing and other articles, features, and advantages of the invention will be apparent from the following more detailed description of the preferred embodiments of the invention. The various features may be utilized or claimed alone or in any combination.

### DESCRIPTION OF THE INVENTION

In the following description, numerous specific details are set forth in order to provide a more thorough description of the present disclosure. It will be apparent, however, to one skilled in the art, that the present disclosure may be practiced without these specific details. In other instances, well-known features have not been described in detail so as not to obscure the disclosure.

In the Summary above, the Description of the Disclosure, and the Claims and Abstract below, reference may be made to particular features (including method steps) of the disclosure. It is to be understood that this disclosure includes possible combinations of such particular features. For example, where a particular feature is disclosed in the context of a particular aspect or embodiment of the disclosure, or a particular claim, that feature may also be used, to the extent possible, in combination with and/or in the context of other particular aspects and embodiments of the disclosure, and in the disclosure generally.

The term comprises and grammatical equivalents thereof are used herein to mean that other components, ingredients, steps etc. are optionally present. For example, an article “comprising11 (or “which comprises”) components A, B and C can consist of (i.e. contain only) components A, B and C, or can contain not only components A, B and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number or the indefinite article “a” (meaning “one”) is used herein to denote the start of a range beginning with that number (which may be a range having an upper limit or no upper limit, depending on the variable being defined). For example “at least one” or “at least a” means 1 or more than 1 The term “at most” followed by a number is used herein to denote the end of a range ending with that number (which may be a range having 1 or 0 as its lower limit or a range having no lower limit, depending upon the variable being defined). For example, “at most 4” means 4 or less than 4, and “at most 40%” means 40% or less than 40%. If, in this disclosure, a range is given as “(a first number), to (a second number)” or “(a first number)-(a second number)”, this means a range whose lower limit is the first number and whose upper limit is the second number. For example, 0-10 mm means a range whose lower limit is 0 mm, and whose upper limit is 10 mm.

The term “or” is used herein as a conjunction used to link alternatives in a series of alternatives. The term “and/or” is used herein as a conjunction meaning that either or both of two options may be valid.

The term “disclosure” may be used interchangeably with the term “invention” in this disclosure. As is well known, only allowed claims constitute an “invention”, even though the course of litigation may depend on such distinctions in terminology. Further, although this disclosure may appear to have a limited scope it is understood that such apparent limitations are merely illustrative of a broader scope of the current disclosure.

Thus, it will be readily understood that the disclosure herein is intended as a framework to describe a general approach to providing appropriate pro Iiposome (see definition below) dosage forms of active ingredients (whether synthetic API's, substantially natural API's and/or derivatives thereof and mixtures and the like, in combination with by one or more appropriate liposomes thereof that would be released according to defined pharmacokinetics.

### Technical Terminology

The following technical terms which are commonly understood by a person skilled in the art may be used in this disclosure:

Active Ingredient: One or more components of a formulation that may provide a medicinal benefit when consumed (see Dosage Form below).

Aerosol: A formulated dosage form that may be temporarily held in a container from which it can be dispensed under pressure by a propellant as a spray. The propellant may be air or any suitable gaseous medium that does substantially no harm to an individual receiving a suspension or a solid or liquid formulated dosage forms carried by the propellant.

Allopathic Supplement/Ingredient: Conventionally accepted medical ingredient (as sanctioned by current Governmental regulations and societal dictates) as opposed to Homeopathic supplement/ingredient (see “description below), which is based on an ancient maxim called “the law of similars” but follows the medical principle of creating “no harm”. The term “allopathic” was coined by Dr. Samuel Hahnemann to distinguish that an allopathic ingredient's efficacy may be governed by the “principle of curing disease by administering substances that produce the opposite effect of the disease when given to a healthy human”. Allopathic ingredients allegedly undergo rigorous clinical testing to prove their efficacy. However, given the complexity of clinical testing, occasionally an allopathic ingredient is later found to have unexpected side-effects. One classic example of an “allopathic” ingredient is thalidomide, which was administered to pregnant women in the 960's but later found to cause birth defects in pregnant women, and is currently used in treating multiple myeloma, but is no longer prescribed for pregnant women. Without limiting this disclosure, it is understood that there is ongoing development of allopathic ingredients, which are regularly updated, and the dosage form of this disclosure may comprise one or more newly developing and unidentified allopathic supplements/ingredients.

Alimentary System: Any portion of the digestive system comprising one or more tubular passages between the mouth (see also the term “Sublingual”) and the anus, including the organs through which food passes for digestion and elimination as waste. Chapter 24 of a book (Principles of Biochemistry by Albert L. Lehninger as discussed below) discusses Digestion, Transport and the Integration of Metabolism in great detail. It should be noted that each organ of the alimentary system may basically be thought of as a portion of a food processing factory, in which certain pH's enzymes and other ingredients are introduced, while certain organs provide mechanical forces and substantially squeeze out water to be processed by the kidneys, and in which certain toxins may be removed by the liver. In fact certain bile acids and salts come from a portion of the lymphatic system (such as the gall bladder. Page 684, FIG. 24-1,page 691, FIG. 24-7,page 691, FIG. 24-8,page 704, FIG. 24-18 of Lehninger are most informative.

Amphipathic (also known as Amphiphilic) Molecule: A molecule including both hydrophobic and hydrophilic groups. The hydrophobic group may be a long carbon chain, with the form CH3(CH2)n/ where n may be any integer and wherein the chain may be straight or branched. The hydrophilic group may comprise one or more suitable elements such as nitrogen, oxygen, sulfur, phosphorus and the like configured in forms such as substituted and unsubstituted amines and their salts, substituted and unsubstituted amides, ureas, alcohols, carboxylic acids and their salts, thiols, phosporous acids, phosphoric acids and their salts and the like.

Amphiphile: A chemical compound possessing both hydrophilic and hydrophobic nature.

Aqueous: A solution of an ingredient in water, such as “aqueous ethanol” meaning a solution of water and ethanol.

Bodily System: Pertaining to all systems identified in living beings such as humans. Thus, the circulatory system relates to blood flow through veins and arteries and passes through organs such as the heart and feeds into the lungs and brain, as well as the kidneys, liver, pancreas, spleen, various glands and so on. Another system is called the lymph system and relates to a fluid containing white cells, chiefly lymphocytes, that is drained from tissue spaces by the vessels of the lymphatic system. The lymph system can transport both aerobic and anaerobic bacteria, fungi, viruses, and cancer cells. It is understood that there are many bodily systems and a variety of synthetic pharmaceutically active ingredients as well as substantially non-synthetic pharmaceutically active ingredients (often termed “natural products” derived from various natural sources) that may be used in treating various bodily systems (see also Immune System). As used herein, such synthetic active pharmaceutical ingredients as well as natural products derived from various sources may be formulated as dosage forms, and may be abbreviated as API's in certain portions of this disclosure as a matter of convenience when related specifically to synthetic pharmaceutically active ingredients.

Capsule: Usually a substantially cylindrical soluble container enclosing a dosage form (see below). Of course, the shape of the capsule may be varied as desired.

Dosage Form: An oral or sublingual tablet, capsule or aerosol which has been medicated with a predetermined amount of one or more ingredients that are active (see above). Examples include one or more homeopathic ingredients or one or more suitable allopathic (non-homeopathic) active ingredients often termed nutritional supplements and/or combinations thereof. While various allopathic ingredients suitable for use in this disclosure may have the suffix USP (United States Pharmacopeia) or NF (National Formulary), this provides for better identification of the ingredient, or its purity, and should not be construed as a limitation of this disclosure, since identical ingredients are available under other designations in other countries.

Excipient: A substantially inert ingredient (see description below) combined with a medicinally active ingredient to facilitate tablet production. Examples may include one or more of the following: binders to insure tablet integrity, disintegrants to permit more rapid access to a medicinally active ingredient, fillers (or diluents) to increase tablet bulk, flow-controlling agents such as lubricants, glidants and anti-adherents, taste enhancers or flavorings such as sweeteners, and colorants to distinguish types of tablets for facile identification by producers and consumers. Excipients enhance administration of a medicinally active ingredient, which often represents a small portion of a tablet's bulk.

Fatty Acids: Unbranched hydrocarbon chains having one or more carboxylic acid groups, connected by single bonds alone (saturated fatty acids) in the chain or by both single and double bonds (unsaturated fatty acids) in the chain.

Glyceride: Esters formed from an organic molecule denoted “glycerol” and one or more fatty acids.

Homeopathic Supplement/Ingredient: A supplement/ingredient defined as shown in the Homeopathic Pharmacopoeia of the United States Revision Service (HPRS) December 2003. Without limiting this disclosure it is understood that the HPRS is regularly updated, and the dosage form of this disclosure may comprise one or more newly developing and as yet unidentified homeopathic supplements/ingredients.

Hydrophilic: Strong affinity for water.

Hydrophobic: Weak affinity for water.

Immune System: The interacting combination of all the body ways of recognizing cells, tissues, objects, and organisms that are not part of itself, and initiating the immune response to fight them. Many organs may be associated with the Immune System, and major research efforts are attempting to understand and correlate organ functionality as manifested in various cells and more particularly at the molecular biochemical level in areas of technology such as bioinformatics, proteomics (study of protein in genes) and molecular biology at the level of RNA (ribonucleic acids) and DNA (deoxyribonucleic acids), hormones, coenzymes and the like (see, for example, Liposomes, and in particular the book by Albert L. Lehninger as discussed below). This may have important applicability to various auto-immune diseases (for example AIDS being one extremely well-known type) and cancer, and may also play a role in the study of cardiology and immunology as well as many other well-known “illnesses” associated with treatment by surgery or alternatively radiation, or targeted as well as systemic chemotherapy with various levels of success depending on the early detection of the illness and, of course, appropriately unbiased and proper diagnosis.

Inert Ingredient: One or more components of a formulation that generally provide no medicinal benefit and generally have substantially no toxic effect when consumed (see Excipient above).

Lipid: A class of hydrocarbon-containing organic compounds essential for the structure and function of living cells which are usually characterized as substantially water-insoluble and substantially soluble in non-polar (or oleophilic) organic solvents (such as ether and the like).

When used as a synonym to the word “fat”, lipids substantially comprise one or more glycerides.

Lipophilic: Strong affinity for fats or other lipids.

Miscible: When referring to a mixture of two fluid components having distinct physical phase appearances, this is the capability of mixing the two physical phases in any ratio without separation of the two phases. The mixture formed by a miscible liquid or solid is described as a solution. Two physical fluid components may be partially miscible with one another (that is a portion of a first physical phase may appear to be a solution in a second physical phase while another portion of the first physical phase may appear to be insoluble in the second physical phase).

Liposome: A very simplified definition is an artificially formed sac or vesicle (structural enclosure element) comprising one or more layers of lipids often used medicinally to carry one or more drugs, vaccines and/or enzymes to one or more targeted cells in a living body. In another aspect, a spherical vesicle with a membrane comprising a phospholipid bilayer used to deliver drugs or genetic material into a cell. Liposomes may comprise naturally derived phospholipids with mixed lipid chains (for example, egg phosphatidylethanolamine), or of pure components such as DOPE (dioleolylphosphatidylethanolamine) and the like as is understood in the art. A good reference that generally describes liposomes and their important biochemical role in cells is “Principles of Biochemistry”, Author Albert L. Lehninger, Chapter 12 (Lipids and Membranes), pages 303-332 (first printed and published April 1982 by Worth Publishers, Inc. NY, N.Y.) having an ISBN: 087901-136-X, which may be incorporated by reference in most aspects of the current disclosure.

Lyophilize: To freeze-dry.

Oleophilic: Strong affinity for oils.

Organic: Being, containing, or relating to carbon compounds, especially in which hydrogen is attached to carbon whether derived from living organisms or not.

Organic Solvent: A solvent including a carbon compound. Examples include, without limitation, glycerin, ethanol and many others as is understood in the art.

Pharmacokinetics: A field of research of the body's reaction to drugs, including their absorption, metabolism, and elimination. A book entitled “Liposome Drug Delivery Systems” by Guru V. Betaaeri, Ph.D, Scott A. Jenkins, B. S. and Daniel L. Parsons, Ph.D at the Division of Pharmaceutics, Department of Pharmacal Sciences the School of Pharmacy Auburn University Published by Technomic Pulbishing Co. r Inc. Copyright 1993 Technomic Publishing Company, Inc. Library of Congress catalog card No. 93-60519 ISBN No. 1 -56676-030-5 addresses some of these issues. Of most interest, is a section found between pages 55 and 56, which is entitled “EFFECT OF LIPOSOME COMPOSITION UPON PHARMACOKINETICS”. Certain generalizations found in this section are particularly interesting because as pointed out “negatively charged small liposomes are cleared more rapidly than neutral or positively charged small liposomes. This increased rate of clearance may be due to an interaction of the negatively charged liposome membrane with plasma components. The increased clearance of phosphatidylserine-containing liposomes may be due to a specific recognition mechanism associated with the scavenging of erythrocytes. It appears that the brain, spleen and bone marrow have greater uptake of positively charged SUV (small unilamellar vesicles), while the lung has greater uptake of negatively charged SUVs”. Further, in this section: “Large negatively charged liposomes are generally cleared at a greater rate by the spleen, bone marrow and monocytes than neutral or positively charged liposomes. The rate of removal is negative>neutral>positive. The rate of uptake by the liver is greatest for neutral liposomes with the rates for negatively and positively charged liposomes being equal. There is evidence of a preferential uptake of both positive and, negative liposomes by the lung”. And even further is stated: “It should be noted that liposomes acquire a negative charge in blood due to an interaction with plasma components. The Iiposome charge may determine which proteins bind to the Iiposome, and these bound proteins may, in turn, determine the fate of the Iiposome. The incorporation of cholesterol, sialogangliosides and cardiolipin into liposomes retards liver uptake, while the incorporation for glyceroceramide enhances liver uptake. In addition, transient lung accumulation is common if there are carbohydrate moieties on the Iiposome surface”. And yet further is stated: “Plasma high-density lipoproteins (HDL) may disrupt liposomes and lead to leakage. The uptake of liposomal lipid by HDL is saturable. HDL removal of phospholipids is facilitated by plasma proteins that adsorb to liposomes and possibly loosen the packing of the bilayer. The susceptibility of liposomes to HDL damage increases with a decrease in Iiposome size as the bilayer becomes more curved. Other lipoproteins have no significant effect on Iiposome stability. Albumin binds to liposomes, but does not cause damage. Incorporation of cholesterol into liposomes tightens the packing of phospholipid molecules in the bilayer. Cholesterol decreases Iiposome interaction with proteins, including protein penetration into the bilayer, and decreases Iiposome susceptibility to phospholipase action. Lipoproteins readily accept liposomal cholesterol. Liposomes may elicit an antibody response, and repeated injections may lead to the onset of antibody-induced complement lysis and release of Iiposome contents”. Finally, the section summarizes all of the above in the following: “The effect of Iiposome dose and charge on Iiposome pharmacokinetics can be summarized as follows: (1) The half-life of liposomes in the blood can be prolonged by RES (Reticuloendothelial System) blockade; (2) The uptake of liposomes by the spleen generally increases after blockade of the liver; (3) Large liposomes appear to have a more pronounced dose effect than small liposomes; (4) Some of the clearance sites for small liposomes cannot be blocked by large liposomes; (5) The effects of liposomal dose on blood clearance and tissue uptake diminish with time, as uptake recovers to normal levels; (6) RES blockade does not seem to increase the uptake of large liposomes by non-RES tissues, although the relative distribution of large liposomes among the RES tissues may be altered; and (7) Charge liposomes should not be use to block the RES”. In fact, the book by Guru V. Betaaeri, Ph.D, Scott A. Jenkins, B. S. and Daniel L. Parsons as described above may be incorporated by reference with respect to most aspects of the current disclosure.

Phospholipids (also known as Glycerophospholipids): Organic molecules comprising a glycerol core esterified with two fatty acid “tails” or chains and having one “head 11 group comprising a phosphate ester linkage. The chains are usually 10-24 carbon groups long, but may be shorter or longer. The head groups of the phospholipids found in many biological membranes include phosphatidylcholine (lecithin), phosphatidylethanolamine, phosphatidylserine and phosphatidylinositol, whose head group can be chemically modified by the addition of one to three more phosphate groups. While phospholipids are the major component of biological membranes, other lipid components like sphingolipids and sterols (such as cholesterol in animal cell membranes) are also found in biological membranes.

Prodrug: A pharmacological substance, which may be administered in an inactive (or significantly less active) form and may be metabolized in the body in vivo into an active compound.

Pro-liposome (pro Iiposome) Formulation: A mixture comprising one or more phospholipids and one or more dosage forms wherein the dosage forms have not been necessarily pre-encapsulated prior to formation into a tablet, capsule or aerosol or other appropriate delivery form. In some aspects, the mixture may compromise other fat soluble components as well as the above-mentioned phospholipids as desired. Advantages of such Pro-liposome (also designated pro Iiposome) formulation are readily apparent as disclosed herein.

Solution: A solid, liquid, or gas mixed homogeneously with a liquid.

Solvent: A substance capable of (or of being used in) dissolving or dispersing one or more other substances (solutes), especially a liquid component of a solution present in greater amount than the solute.

Sublingual: One or more medicines or dosage forms (see above) that may be administered by being placed under the tongue or in an oral cavity to dissolve with saliva. As used herein, for a non-pre-encapsulated (or pro Iiposome) tablet having one or more phosopholipid ingredients/components, one or more dosage forms, one or more excipients and one or more other medicinally non-active ingredients/components, formation of one or more encapsulated Iiposome comprising one or more phospholipids and one or more dosage forms may occur when placed under the tongue or in the oral cavity.

Surfactant: In general, a surfactant is a substance useful for its cleansing, wetting, dispersing, or similar powers. Each of such functions is performed by a surfactant due to its ability to reduce surface tension. More specifically, as used herein, a surfactant is an ingredient that reduces the surface tension of water. With respect to this disclosure, aspects of Iiposome structure may include the properties of surfactants as described herein (see miscible above).

Suspension: Particles mixed in a fluid or a solid which may not be dissolved (or remain separated) in the fluid.

Tablet: As used herein includes tablets of any shape and caplets (which may be tablets having a capsule shape).

Topical Formulation: Medications that are applied directly to the surface of the part of the body being treated, but may travel to other organs such as may occur with patches such as nicotine patches, lidocaine patches and the like where transportation rates through the skin may determine the dosage as well as how long the dosage is effective and may be controlled by various means associated with formulation and environmental factors at least.

Water Miscible Organic Solvent: An organic solvent that can be mixed with water in any ratio without separation of the water from the organic solvent. In the practice of the invention, some examples (but not required) water miscible organic solvents include ethanol and similar solvents that are commonly used in FDA food regulated applications.

A better understanding of the relevance of the following disclosure may be better understood by reference to a review article entitled “Laboratory Drug Delivery System from Laboratory to Clinic” authored by Kshirsager, N. A. et al. (see www.j pgmonline.com) and published in the Journal Of Postgraduate Medicine, 2005, Vol. 51, Supplement 1, pages S5-S15. This specific publication describes the development of an intravenously delivered liposomal dosage form and describes the difficulties associated with economically commercializing such a dosage for. As is well known, patients are placed at greater risk when invasive methods such as intravenous delivery have been used. In this case, the liposomal dosage form comprises an antifungal API.

As will be appreciated and as described below, an advantage of the Pro-liposome Formulation Systems described herein may be that they can be formulated to be non-invasive, thus leading to diminished side-effects during treatment.

Description of Some Exemplary Active Pharmaceutical

Ingredients

Without limiting the disclosure, as defined herein, Active Pharmaceutical Ingredients (API) may include any known synthetic pharmaceuticals and derivates thereof and may further include substantially non-synthetic natural products or derivatives thereof, and of course any suitable combinations thereof as desired. Many examples of these products exist and some are readily available in the Orange Book of Approved Drug Products with Therapeutic Equivalence Evaluations. According to the first page, the latest 28^{th} Edition (2008) states: “The products in this list have been approved under Section 505 of the Federal Food, Drug, and Cosmetic Act. The Orange Book is regularly updated by the U.S. Department of Health and Human Services Food and Drug Administration Center for Drug Evaluation and Research Office of Pharmaceutical Science Office of Generic Drugs. Other sources of known synthetic drugs may be found in similar registries published worldwide.

Furthermore, the worldwide patent literature also describes multiple synthetic drugs and Table 1 below shows just a small sampling of some of the API's of greatest economic value in 2007 with their corresponding CAS Registry Numbers. As is well-known, the CAS Registry may be the largest and most current database of chemical substance information in the world, containing more than 34 million organic and inorganic substances and 59 million sequences. The CAS Registry covers substances identified from the scientific literature from 1957 to the present, with additional substances going back to the early 1900s. Extracted natural products are also listed in the CAS. Of course, the CAS Registry may be updated regularly too.

Additionally, and without limiting the disclosure, the U.S. National Center for Natural Products Research, may provide resources for sourcing of natural API's as may any literature freely available on searching multiple web sites and open literature as desired. By and large, the current patent literature often discloses the direction in which multiple entities have directed their research efforts to produce API's as provided by the pharmaceutical and biotech industries by and large.

TABLE 1
List Of Top Selling Retail Brand Drugs (in U.S. Dollars) in 2007

| No. | Name OF API | CAS Registery Number |
|---|---|---|
| 1 | ATORVASTATIN | 134523-00-5 |
| | ESOMEPRAZOLE | 161796-78-7 |
| 3 | FLUTICASONE | 80474-14-2 |
| | SALMETEROL | 89365-50-4 |
| 4 | LANSOPRAZOLE | 103577-45-3 |
| 5 | CLOPIDOGREL | 113665-84-2 |
| | MONTELUKAS | 158966-92-8 |
| 7 | QUETIAPINE FUMARATE | 111974-69-7 |
| 8 | VENLAFAXINE | 93413-69-5 |
| 9 | ESCITALOPRAN OXALATE | 128196-01-0 |
| 10 | PIOGLITAZONE. | 111025-46-8 |

Description of Some Exemplary Pro-Liposome Oral Tablet Forms

Without limiting the disclosure, a pro-liposome oral tablet having a dosage form of this disclosure may comprise one or more fillers (or carriers) including at least one of anhydrous and hydrated forms of a sugar and a sugar alcohol selected from the group consisting of lactose, dextrose, sucrose, fructose, maltose, xylose, maltodextrin, dextrin, cyclodextrin, mannitol and sorbitol, and the like as is understood in the art Additionally, the oral tablet may further comprise one or more fillers such as calcium phosphate and the like. In an embodiment of the current disclosure, fillers may comprise about 60% to about 90% by weight of the oral tablet. However, naturally, the amount of fillers may be altered according to the needs of an appropriate formulation of an oral tablet according to the current disclosure. The sugar and the sugar alcohol may provide both a pleasant taste and may provide for rapid disintegration in any suitable portion of the alimentary system, while acting as a binder, which substantially maintains the shape of the oral tablet prior to ingestion.

Without limiting the disclosure, the pro-liposome oral tablet having the dosage form may further comprise one or more glidants. Exemplary glidants may include talc, at least one form of anhydrous silica and hydrated silica (or silicon dioxide) selected from the group consisting of precipitated, fumed, amorphous, colloidal, crystalline, and the like. Of course, any glidants currently known or that may be developed in the future may be suitably incorporated in an oral tablet of the current disclosure. In an embodiment of the current disclosure, glidants may comprise about 0.1% to about 2.0% by weight of the oral tablet. However, it is understood that the amount of glidants may be altered according to the needs of an appropriate formulation of an oral tablet according to the current disclosure. Silica may provide both some degree of flow control, and increases the hardness of the tablet.

Without limiting the disclosure, the pro-liposome oral (or sublingual) tablet having the dosage form may further comprise at least one substantially hydrophobic lubricant including stearic acid, one or more alkali metal salts of a fatty acid selected from the group consisting of calcium stearate and magnesium stearate, and the like. Of course, the fatty acid may be any GRAS approved fatty acid. GRAS ingredients are “Generally Regarded As Safe” by the EPA, FDA or any other governmental agency responsible for setting guidelines regarding ingredients ingested by humans as is understood in the art. Additional lubricants may include or more vegetable oils or one or more polyethylene glycols suitable for ingestion as understood in the art. Of course, any lubricants currently known or that may be developed in the future may be suitably incorporated in an oral tablet of the current disclosure. In an embodiment of the current disclosure, lubricants may comprise about 0.1% to about 5% by weight of the oral tablet having the dosage form. However, it is understood that the amount of lubricants may be altered according to the needs of an appropriate formulation of an oral tablet according to the current disclosure.

Without limiting the disclosure, the pro Iiposome oral tablet having the dosage form may comprise at least one disintegrating agent (disintegrant or co-disintegrant since the one or more fillers described above may also function as disintegrants). However, in the context of this disclosure one or more of the co-disintegrants (disintegrating agents) may not as rapidly absorb water as one or more of the fillers described above, but rather may require appropriate environmental conditions before disintegrating. Without limiting the disclosure, an example of the function of such a disintegrating agent may be a slow release tablet. Without limiting the disclosure, co-disintegrants may include one or more of the following: starch, crosscarmelose, crospovidone, sodium starch glycolate, micro crystalline cellulose, lactose, dextrose, gelatin, gum acacia, stearic acid, calcium phosphate, alginic acid, calcium carboxymethylcellulose, sodium carboxymethylcellulose, methylcellulose, hydroxyethylcellulose, hydroxypropylcellulose, hydroxypropylmethylcellulose phthalate, non-crystalline cellulose, polyvinyl alcohol, or polyvinyl pyrrolidone and the like. As disclosed herein, the disintegrating agent may be rapidly water soluble or may be configured to disintegrate gradually in any portion of the alimentary system. Generally, a disintegrant does not substantially add to the grittiness of the oral tablet dosage form when ingested. Of course, any disintegrants currently known or that may be developed in the future may be suitably incorporated in an oral tablet of the current disclosure. In an embodiment of the current disclosure, co-disintegrants may comprise about 1% to about 20% by weight of an oral tablet having the dosage form. However, it is understood that the amount of disintegrants may be altered according to the needs of an appropriate formulation of an oral (or sublingual) tablet according to the current disclosure.

Numerous Iiposome drug (or active ingredient) delivery systems are known and have been characterized. Such Iiposome systems comprise phospholipids that pre-encapsulate the dosage forms and may be prepared from lysophosphatides in the presence of equimolar cholesterol and other single chain amphiphiles. The most common phospholipids are phosphatidylcholines which are amphipathic molecules having a glycerol bridge linking a pair of hydrophobic acyl hydrocarbon chains with a hydrophilic polar head group (phosphocholine). It is thought that phosphatidylcholines are substantially insoluble in aqueous environments and that phosphatidylcholines align closely to form substantially planar bilayer sheets with long chain hydrocarbon fatty acid chains of the phosphatidylcholines interacting between the bilayers and an outer portion of the bilayers consisting of hydrophilic components of the phosphatidylcholines thereby minimizing unfavorable interactions between aqueous environments and the long chain hydrocarbon fatty acids. Common phosphatidylcholines are also known as lethecin and can be derived from both natural and synthetic sources such as egg yolks, soya bean, bovine heart and spinal cord tissue. Lethecin obtained from plant versus animal sources have different compositions and more specifically plant lethecin often has a higher level of polyunsaturated fatty acyl chains compared to animal lethecin which often comprises a higher proportion of fully saturated fatty acid chains.

A method of classifying liposomes depends on the size of vesicles formed during the manufacture of liposomes, which may differ greatly according to the procedure used. In one aspect, liposomes are classified as multilamellar vesicles (MLVs). In another aspect liposomes may comprise unilamellar vesicles (UVs) which may be subdivided into two classes, namely, small unilamellar vesicles (SUVs) having a vesicle size of under 100 nm (nanometers) and large unilamellar vesicles (LUVs), which are greater than about 100 nm in size.

SUVs are generally optically clear combinations (or preparations) comprising a bilayer of phospholipid surrounding an aqueous space (or solvent). It has been thought that SUVs have a low encapsulation efficiency (0.1-1% depending on the lipid concentration). SUVs are generally spherical in shape with a minimum radius of about 20 nm.

MLVs generally have lower encapsulation capacity defined as liters (1) of aqueous space per mole of lipid (1-4 l/mole). MLVs have less encapsulation capacity than LUVs of 25 comparable size. MLVs range from 100 nm to 1000 nm (or 1 micron) or more in size and generally comprise five or more lamellae (layers).

Without limiting the disclosure, in an exemplary embodiment a pro-liposome oral tablet having the dosage form may comprise one or more highly purified phospholipids and derivatives thereof configured to encapsulate one or more active ingredients in a portion of the alimentary system including under the tongue (or sublingually) or any oral cavity where, for example, saliva may activate Iiposome formation. The one or more phospholipid derivatives may react with water depending on the pH (or hydrogen ion concentration), salt concentration (or electrolyte concentration), metabolic conditions and the like of the alimentary system environment to provide vesicles configured to encapsulate the nutritional supplements of the dosage form in situ rather than being pre-formed prior to ingestion. For example, the portion of the alimentary system may comprise a portion of the stomach wherein the pH is substantially acidic because of production of hydrochloric acid.

In an exemplary embodiment, a suitable water-soluble phospholipid may comprise SOLUTHIN® Ca (a proprietary highly purified phosphatidylcholine carried on a substantially calcium lactate compound comprising a minimum of about 50% by weight phosphatidylcholine as a powder form available from the American Lecithin Company (USA)). In an alternative exemplary embodiment, a suitable water-soluble phospholipid may comprise SOLUTHIN® MD (a proprietary highly purified phosphatidylcholine on a substantially maltodextrin compound comprising a minimum of about 20% by weight of phosphatidylcholine as a powder form available from the American Lecithin Company (USA) and elsewhere). Furthermore, a portion of either SOLUTHIN® Ca or SOLUTHIN® MD may comprise Lysophosphatidylcholine with a maximum by weight of about 6% of the SOLUTHIN®.

The size of vesicles in an aqueous dispersion of SOLUTHIN® Ca, may be less than about 200 nm (nanometer) as determined analytically. In some aspects, the vesicle sizes may be between about 100 nm (nanometer) to about 200 nm (nanometer) in size as determined analytically. A portion of the phosphatidylcholine may remain complexed with the calcium compound and may be observed as a sediment in an aqueous dispersion. Furthermore, at least a portion of the phosphatidylcholine may be complexed and vesiculated, depending on various factors including but not limited to pH, salt (electrolyte) concentration and numerous other factors and the like.

Of course, any combination of phospholipids or derivatives thereof currently known or that may be developed in the future may be suitably incorporated in an oral tablet of the current disclosure. Further, without limiting the disclosure,clearly any suitable phosphatidylcholines, combination of other liposomes and other co-products whether synthetic or extracted from natural sources may be configured in powder form on any suitable carriers as is well understood in the art and may be adapted to be used as a means of targeting specifc bodily organs according to a desired characteristic of the carrier.

Of course, without limiting the current disclosure one or more phosphatidylcholine and/or phosphatidylserine or any other suitable liposomes may be sprayed (fluid bed drive) directly onto an active pharmaceutical ingredient and dried according to this disclosure or directly sprayed and dried onto any suitable carrier to provide a powder form suitable for further formulation according to one or more aspects of this disclosure to provide a suitable pro Iiposome system. Without limiting the disclosure some carriers may include di-calcium phosphate, maltodextrin, cyclodextrins, various poly-saccharides or any other substantially non-toxic carriers that would provide a powder form as desired in formulating a pro-liposome formulation system.

In an embodiment of the current disclosure, one or more phospholipids and/or derivatives of phospholipids may comprise about 1% to about 30% by weight of a pro-liposome oral tablet (or as a capsule or aerosol formulation as discussed below). However, it is understood that the amount of phospholipids and phospholipid derivates thereof may be altered according to the needs of an appropriate formulation of an oral tablet having one or more API's, natural API's and combinations thereof according to the current disclosure.

Without limiting the disclosure, the concentrations of SOLUTHIN® Ca and SOLUTHIN® MD may be in a formulation range of up to about 80% for each individually, or as combinations of each of SOLUTHIN® Ca and SOLUTHIN® MD as desired. It should be noted again that SOLUTHIN® Ca may have a phosphatidyl choline concentration in the range of about 50-60% while SOLUTHIN® MD may have a phosphatidyl choline concentration in the range of about 20-30%.

Without limiting the disclosure, the dosage form may have one or more combinations of any suitable active ingredients (APIs, natural API's or combinations thereof). In an exemplary embodiment of a dosage form configured as a homeopathic ingredient, this dosage form may comprise one or more homeopathic human growth hormones (hGH) which is alternatively termed somatotropin herein with a dilution level of about 28 times in one or more suitable hydrophilic solvents such as water, ethanol and the like and combinations thereof as is understood in the art of homeopathic medicinal ingredient preparation. Of course a homeopathic dosage form may comprise less or greater dilutions of somatotropin in one or more suitable solvents.

Somatotropin is a growth hormone (GH) comprising 191 amino acid single chain polypetitides which is naturally synthesized, stored and secreted by the stomatotraph cells within the lateral wings of an anterior pituitary gland and may be extracted and cultivated from various animal sources (bovine being one of the most well documented) as is understood in the art. Synthetic versions of somatotropin such as “Humatrope” are also available from companies such as Eli Lilly Company (USA). As understood in the art yet another source of synthetic somatotropin is available form Genenetech (USA) via recombinant DNA techniques. In Europe, several Corporations such as Pharmacia (formerly Kabi and now Pfizer), Novo, and Serono market nearly identical synthetic human growth hormone as that available from Eli Lilly Company. In one or more embodiments the dosage form may comprise allopathic or homeopathic versions of bio-similar medicines such as interferon alpha, interferon beta, insulin, erythropoietin, and granulocyte-colony stimulating factor. Somatotropin may stimulate growth and cell production in humans and other vertebrate animals.

In an exemplary embodiment, a pro-liposome sublingual tablet may comprise one or more of L-Glutathione, derivatives and prodrugs thereof and flavor enhancers. In yet another exemplary embodiment a sublingual tablet of a pro-liposome formulation may comprise ascorbic acid (Vitamin C), ascorbates or esters of ascorbic acid, prodrugs comprising ascorbic acid and combinations thereof in addition to flavor enhancers. In yet another exemplary embodiment, an orally ingested pro-Iiposome tablet may comprise an active ingredient of one of the following: L-Glutathione, ascorbic acid, ascorbates, esters of ascorbic acid and prodrugs masking as any of these compounds, which may be metabolized into these compounds.

Of course, the dosage forms described herein may comprise synthetic or natural active ingredients, such as steroids, vitamins, minerals, and the like as is understood in the art, which may be incorporated in the pro-liposome oral tablet (or capsules or aerosols) of the current disclosure. Furthermore, it is understood that the amount of active ingredient may be varied according to any specific targeted value. Thus homeopathy deals with various dilutions of active ingredients as is well understood in the art. In an exemplary embodiment of the oral tablet wherein the dosage form includes somatotropin at about a 28 times dilution in water, the active ingredient may comprise about 1% to about 10% by weight of the oral tablet. However, it is understood that the amount of active ingredients thereof may be altered according to the needs of an appropriate formulation of an oral tablet according to the current disclosure.

In an exemplary embodiment ingredients (or components) of a pro-liposome oral tablet may comprise about 74% by weight of a mixture of at least one of the following fillers: lactose, dextrose, calcium, phosphate, maltodextrin, cyclodextrin, sorbitol, about 1% by weight of a homeopathic active ingredient (see description above), about 17% by weight of co-disintegrants (see description above), about 2% by 20 weight of lubricants (see description above), about 1% by weight of glidants (see description above) and about 5% by weight of phospholipids and derivates thereof. In another embodiment of an oral tablet, the tablet may comprise about 77% by weight excipients, about 17% by weight co-disintegrants, about 1% by weight of one or more active ingredients and about 5% by weight of one or more phospholipids and one or more derivates thereof.

Of course, without limiting the disclosure, any tablet formulations described herein could also be configured as capsule formulations. In an aspect of the disclosure, the Iiposome delivery systems (oral dosage forms) described herein may change the half life of one or more active ingredients depending on formulation considerations and the nature of the capsule. Naturally, a capsule may provide yet another method of targeting specific bodily locations to release one or more API's or natural API's or combinations thereof having an appropriate Iiposome (for example, SOLUTHIN® Ca, SOLUTHIN® MD or combinations thereof and other suitable liposomes and ingredients as desired). Such art is known for example in the form of time-released dosage forms. It will be further appreciated that the ingredients of an outer capsule may be altered as desired, even though most capsules follow certain well-defined and commonly available formulations. Thus, the nature of the capsules may be merely a design choice. As desired a formulator may alter the capsule features (such as by thickness of the side walls, ability of the capsule to dissolve in a specific portion of the alimentary system, pH and the like). Naturally, the appropriateness of a capsule may be determined according to where it was desired to pharmacokinetically release a combination of liposomes and API's (synthetic or natural and/or combinations thereof).

In yet another aspect of the disclosure a pro-liposome capsule may have a higher percentage of phosphatidylcholine liposomes enclosed therein compared to a tablet. Without limiting the disclosure, for example, about 90% of a capsule encased dosage form may comprise phosphatidylcholine liposomes and the Iiposome population may be increased by many factors of 10. This may be advantageous, for example, to facilitate penetration of targeted cell membranes of one or more bodily organs and transfer synthetic or substantially natural API's or combinations thereof as desired.

In still another aspect of the disclosure, one or more pro-liposome formulations as described above may be added to an aerosol nasal spray or to an oral spray as desired. Furthermore, pro-liposome formulations may be delivered with one or more vaccines (preparations containing weakened or dead microbes of the kind that may cause a disease and which may be administered to stimulate the immune system to produce antibodies against that disease).

In another aspect of the disclosure a pro-liposome aerosol may comprise a dry powder mist including one or more liposomal dosage forms as described When one or more pro-liposome dosage forms (such as a SOLUTHIN® Ca and/or SOLUTHIN® MD formulations described above) make contact with one or more specifically targeted mucous membranes, Iiposome formation would take place. The liposomal components of one or more of the SOLUTHIN® Ca or SOLUTHIN® MD may be released when exposed to water (or other pre-defined physical condition) and facilitate transport through one or more cell membranes of one or more targeted bodily organs including but not limited to the arterial or venous system and/or the lymphatic system as desired.

Exemplary Method for Producing Some Pro-Liposome Oral Tablets Comprising One or More Liposome Derivatives

The preparation of encapsulated liposomes is well documented in various literature sources. In a commonly known procedure, one or more lipids are prepared for hydration by dissolving and mixing the lipids in an organic solvent such as chloroform or mixtures of chloroform and methanol at 10-20 mg of lipid per ml of organic solvent. The lipids solution may then be converted to a thin lipid film wherein the solvent is removed and the lipids may be frozen and further lyophilized to produce a film or a cake. Hydration of the dry lipid film/cake may be accomplished by adding an aqueous medium to the container of dry lipid and agitating the mixture. The hydration medium should be at a temperature exceeding the gel-liquid crystal transition temperature (Tc or Tm) of the lipid with the highest Tc before adding to the dry lipid. This mixture should be maintained above the Tc during the hydration period. This method may produce LMVs with each lipid bilayer separated by a water layer, wherein spacing between lipid layers is dictated by composition with poly hydrating layers being closer together than highly charged layers. A hydrophobic drug may be co-dissolved with one or more lipids in an organic solvent to produce a homogeneous mixture. The organic solvent may be removed and the lipid/drug residue processed as a typical Iiposome preparation. The size of the LMVs may be further reduced by extrusion, using mechanical energy or sonication techniques.

The following exemplary method differs from the commonly known method described above because the active components or ingredients are not pre-encapsulated as one or more liposomes or Iiposome derivatives. Consequently, many of the difficulties associated with production of oral forms of active ingredients in combination with one or more liposomes may be avoided. Advantageously, there is no need to encapsulate or incorporate liposomes comprising combined active ingredients and one or more phospholipids within yet another capsule or to spray a coating over a combination of active ingredients and one or more lipsomes as is known in the art.

The phospholipids (or derivatives thereof) described herein may comprise a mixture of phosphatidyl ethanolamine, di stearylphosphatidyl choline, dimyristylphosphatidyl choline, phosphatidyl choline, dipalmitoylphosphatidyl glycerol, dipalmitoylphosphatidyl choline, dipalmitoylphosphatidic acid or lecithin. In a step of the method, the phospholipids (described above) may be sprayed onto a carrier substance. The carrier substance may comprise at least one of the following: xylitol, mannitol, arabinose, ribose, xylose, glucose, mannose, galactose, sucrose, lactose, calcium lactate, or calcium phosphate to provide a Iiposome forming compound.

In another step of the method, the Iiposome forming compound (one or more phospholipids and derivatives thereof) may be blended with one or more homeopathic ingredients. Of course, the Iiposome forming compound may be blended with one or more allopathic ingredients and/or a mixture thereof to provide an active Iiposome forming intermediate (hereinafter denoted an ALI). In yet another step, one or more co-disintegrants may be added to the blend of the active Iiposome forming intermediate (ALI) to provide a co-disintegrant active Iiposome forming intermediate (hereinafter designated alternatively as a C-DALI). The co-disintegrants may comprise at least one of the following: starch, crosscarmelose, crospovidone, sodium starch glycolate, micro crystalline cellulose, lactose, dextrose, gelatin, gum acacia, stearic acid, calcium phosphate, alginic acid, calcium carboxymethylcellulose, sodium carboxymethylcellulose, methylcellulose and other carboxymethylcellulose salts thereof as is understood in the art, hydroxyethylcellulose, hydroxypropylcellulose, hydroxypropylmethylcellulose phthalate, non crystalline cellulose, polyvinyl alcohol, or polyvinyl pyrrolidone which are available from a variety of commercially available sources as is known in the art. Of course, these co-disintegrants have suitable physical and chemical characteristics to facilitate appropriate Iiposome production on release in a specific portion of an alimentary system.

Furthermore, co-disintegrants may be selected from the group consisting of alginates, polysorbates, polyethylene glycols, acacia, methylcellulose, tragacanth, guar gum, locust bean gum, carageenan, xanthan, collagen and combinations thereof. Of course, co-disintegrants may also include derivatives of co-disintegrants and combinations thereof. Derivatives of disintegrants may include salts of the first and second group alkali metals or ammonium salts or quaternary amines as is known in the art. By way of example, alginates are salts of alginic acid, which is a polyuronide made up of a sequence of two hexuronic acids: beta-D mannuronic acid and alpha-L guluronic acid extracted from brown seaweeds. Commercially, alginates are often offered as sodium and potassium salts of alginic acid.

In another step of the method, one or more lubricants may be blended with the active Iiposome forming intermediate (ALI) or alternatively the C-DALI (co-disintegrant active Iiposome forming intermediate) to provide a lubricated C-DALI (hereinafter denoted a LC-DALI). The one or more lubricants may comprise at least one of the following: talc, magnesium stearate, calcium stearate, stearic acid, or any stearic acid salts as is understood in the art, hydrogenated vegetable oils as is understood in the art, or polyethylene glycols as is understood in the art and the like. In yet a further step one or more glidants may be blended active Iiposome forming intermediate or with the C-DALI, or with the LC-DALI to provide a feedstock for making an oral tablet according to the disclosure herein. Of course it is understood that the order of blending of active Iiposome forming intermediate (ALI) with the remaining ingredients is inconsequential.

Furthermore, taste modifiers such as sweeteners which may be natural sugar products such as sucrose, fructose, maltose, xylitol, sorbitol, glucose, erythritol, maltodextrin, tagatose, lactose and the like as is understood in the art and combinations thereof and/or synthetic products such as aspartame, neotame, saccharin, acesulfame-K, sucralose and the like as is understood in the art, and combinations thereof may be added to the ALI. Additionally, chelating agents such as disodium ethylene diamine acetic acid (EDTA) and natural acids such as citric acid, alpha lipoic acid and the like may also be ingredients of the subject ALI.

In yet another embodiment suitable prodrugs (see description above) and/or preservatives that are pharmaceutical grade ingredients obtainable from a variety of sources well known in the art may be blended with to provide a pseudo ALI. Without limiting the disclosure, some suitable preservatives include ethanol, benzoate and sorbate salts of alkali metals such as sodium and potassium and the like as is understood in the art.

It will be further appreciated that no specific equipment or conditions are necessary to blend the components to provide a feedstock for the pro-liposome oral tablet of the current disclosure and the equipment used is well understood in the art. Furthermore, the method of converting the feedstock of the oral tablet into a pro-liposome oral tablet may be practiced on commonly known equipment known in the art.

In an exemplary embodiment of a method for manufacturing a sublingual dosage form, the method comprises the steps of providing at least one solvent-borne medicating ingredient in a solvent and providing a substantially inert preformed tablet including at least one excipient, one or more phospholipids and derivatives thereof and at least one co-disintegrant, the at least one co-disintegrant adapted to selectively release the at least one solvent-borne medicating ingredient in a portion of an alimentary system as described above. The method may further comprise the steps of impregnating the inert preformed tablet with the at least one solvent-borne medicating ingredient (or dosage form) and evaporating at least a portion of the solvent from the impregnated inert preformed tablet without disintegrating the inert preformed tablet thereby both retaining the shape while medicating the inert preformed tablet to provide the sublingual dosage form.

In an embodiment of the method, the method of manufacturing the inert tablet comprises the steps of screening at least one of anhydrous and hydrated forms of a sugar and a sugar alcohol through a sieve mesh, followed by screening at least one co-disintegrant through a sieve mesh, adding a solvent to both of the at least one of the sugar and the sugar alcohol and the co-disintegrant and wet granulating both of the at least one of the sugar and the sugar alcohol and the co-disintegrant, drying both of the at least one of the sugar and the sugar alcohol and the co-disintegrant at no more than about 40° C. until both of the at least one of the sugar and the sugar alcohol and the co-disintegrant are substantially solvent free. Both of the at least one of the sugar and the sugar alcohol and the co-disintegrant may be further granulated through a sieve mesh to provide a granulated mixture and silica may be added to the granulated mixture and the silica and the granulated mixture may be screened through a sieve. The method of manufacturing the inert tablet may further comprise the steps of adding at least one alkali metal stearate to the silica and the granulated mixture and screening the at least one alkali metal stearate, the silica and the granulated mixture through a sieve mesh, and blending and compressing the at least one alkali metal stearate, the silica and the granulated mixture thereby providing the inert preformed tablet having a predetermined shape.

In a specific example of manufacturing the inert preformed tablet at least one of lactose, dextrose and sucrose comprising about 9% by weight of the inert preformed tablet is screened through a #18 mesh sieve and Crospovidone (a co-disintegratant) is also screened through a #18 mesh sieve. Both the at least one of lactose, dextrose and sucrose and the Crospovidone comprising about 16.0% by weight of the inert preformed tablet may be wet granulated as a mixture with at least one of ethyl alcohol and isopropyl alcohol, and then the mixture may be placed in a drying oven at a temperature of about 39° C. until the mixture is substantially dry. The dried mixture may be then dry granulated through a #18 sieve. Colloidal silica comprising about 1.4% by weight of the inert preformed tablet may be screened through a #18 sieve and blended with the dried mixture for about minutes. Magnesium stearate comprising about 0.7% by weight of the inert preformed tablet may be also sieved through a #18 sieve and blended together with the at least one of lactose, dextrose and sucrose, the Crospovidone, and the colloidal silica for about 5 minutes thereby providing a powdered form of the inert tablet. A powdered phospholipid (about 5% by weight of a final sublingual tablet) may also be added during this step or any earlier step. The powdered form of the inert tablet may be compressed into an inert preformed tablet. The tablet may have at least one marking showing the degree of strength of the liquid drug attenuation to be used to medicate the inert preformed tablet and identifying the manufacturer. Either of one or more homeopathic ingredients or one or more allopathic ingredients and/or combinations thereof may be dissolved in a solvent of at least one of water, ethyl alcohol and isopropyl alcohol at the desired liquid drug attenuation, and then the solution of the homeopathic ingredients (and/or allopathic ingredients) and the solvent may be sprayed on the surface of the inert preformed tablet and the resulting medicated inert preformed tablet may be dried at a temperature of no more than about 40° C. thereby providing a required sublingual dosage form.

In an alternative embodiment of a method for manufacturing an inert tablet in which no solvent is used to manufacture the inert tablet, the method comprises the steps of screening at least one of a sugar and a sugar alcohol through a sieve mesh and screening at least one co-disintegrant through a sieve mesh; commingling the at least one co-disintegrant with the at least one of the sugar and the sugar alcohol and granulating the at least one co-disintegrant and the at least one of the sugar and the sugar alcohol through a sieve mesh to provide a granulated mixture. The method for manufacturing the inert tablet may further comprise the steps of adding silica to the granulated mixture and screening the silica and the granulated mixture through a sieve, adding at least one alkali metal stearate to the silica and the granulated mixture and screening the at least one alkali metal stearate, the silica and the granulated mixture through a sieve mesh, followed by blending and dry compressing the at least one alkali metal stearate, the silica and the granulated mixture thereby providing the inert preformed tablet having a predetermined shape. Of course a powdered form of one or more phospholipids may be incorporated into any of the previous steps. The inert preformed tablet may be medicated by a number of methods. Typically, in an example of a homeopathic ingredient dosage form, the dosage form may be dissolved in at least one of water, ethyl alcohol and isopropyl alcohol and mixtures thereof, and the like at a predetermined attenuation to form a homeopathic solution. The homeopathic solution may be applied on a portion of the surface of the inert preformed tablet by spraying. Alternatively, the inert preformed tablet may be placed in a container and a homeopathic solution may be added with a predetermined contact time between the homeopathic solution and the inert preformed tablet.

Although the description above illustrates a substantially batch process, it is contemplated that a continuous process may also provide a suitable phospholipid/dosage form (or pro-liposome) product as described herein. Of course, the above process descriptions may be equally applied to allopathic and/or homeopathic and mixtures thereof dosage forms.

It is understood that pro-liposome oral tablets made as described herein may have a variety of uses including as nutritional supplements and as food additives when ingested.

Method of Dosing a Person with a Pro-Liposome Oral Tablet Comprising One or More Phospholipid/Dosage Form

Derivatives

In an embodiment, a therapeutic dose of a pro-liposome oral tablet comprising about 77% excipients, about 17% co-disintegrants, about 1% of one or more active ingredients and about 5% of one or more phospholipids and derivates thereof as described above may be ingested with a liquid such as water. When the oral tablet passes through the alimentary system, a portion of the tablet such as excipients may begin to disintegrate without releasing the one or more phospholipid derivatives and one or more active ingredients. After a local environmental condition is achieved in the alimentary system (as described above), one or more co-disintegrants may dissolve in a portion of the alimentary system to release at least a portion of a combination of one or more active ingredients and one or more phospholipid derivatives and thereby provide one or more liposomes comprising at least a portion of the active ingredient in a portion of the one or more phospholipid derivatives in situ. The in situ encapsulated Iiposome described herein may provide a benefit to the consumer of the oral tablet in comparison with a nonliposome dosage form of an oral tablet.

Of course, any embodiment of the oral tablet described herein may be packaged as is understood in the art.

While the invention has been particularly shown and described with reference to preferred embodiments thereof, it will be understood by those skilled in the art that the foregoing and other changes in form and details may be made therein without departing from the spirit and scope of the invention. For example, a pro-liposome tablet, sublingual tablet, capsule or an aerosol may be considered to be similar variations of pro-liposome formulation systems.

Examples of Oral Dosage form Using Colloidal Liposomal Technology

Endorphamine (Symbiotic Diet)
Ingredients

Thiamine HCL USP
Pyridoxine HCL USP
Pridoxal-5-phosphate
Cr Polynicotinate
(as ChromeMate ®)
10% Elemental Cr
Endorphamine
KB220Zn LTS Complex
DL Phenylalanine (as
D-PhenEZE ™)
L-Tyrosine USP
Rhodiola Rosea
Extract
3% Rosavins Powder
(as RhodiGen ™)
Passion Flora
Incarnata Extract Powder
3.5% Vitexin (as EM103 ™)
5HTP
L-Glutamine
Zehntose
(metallosaccharide
complex containing:
Arabinogalactans (100 mg)
N-acetylglucosamine
(18 mg)
Astragalus (root)
(18 mg)
Aloe Vera (inner leaf)
(18 mg)
Indian Frankincense
Gum Resin (16 mg)
Inulin (10 mg)
White Pine Bark
Extract (14 mg)
Spirulina (6 mg)
Gymnema (25%)
Huperzine A (1%) (as
AlZine ™)
Bauhinia Extract 10:1
(as AS1000 ™)
Vanilla Extract

All ingredients have a wide range from 1 microgram to 50,000 mg

SynaptagenX
Ingredients

Thiamine HCL USP
Pyridoxine HCL USP
Pridoxal-5-phosphate
Cr Polynicotinate
(as ChromeMate ®)
10% Elemental Cr
Endorphamine
KB220Zn LTS Complex
DL Phenylalanine (as
D-PhenEZE ™)
L-Tyrosine USP
Rhodiola Rosea
Extract
3% Rosavins Powder
(as RhodiGen ™)
Passion Flora
Incarnata Extract Powder
3.5% Vitexin (as EM103 ™)
5HTP
L-Glutamine
Zehntose
(metallosaccharide
complex containing:
Arabinogalactans (100 mg)
N-acetylglucosamine
(18 mg)
Astragalus (root)
(18 mg)
Aloe Vera (inner leaf)
(18 mg)
Indian Frankincense
Gum Resin (16 mg)
Inulin (10 mg)
White Pine Bark
Extract (14 mg)
Spirulina (6 mg)

All ingredients have a wide range from 1 microgram to 25,000 mg

Aminogenix*
Biochemical composition of the Amino Acids and
Neuroadaptagen Mixture

| Kilo joule | per 100 g | 8 g/day | biosynthesis |
|---|---|---|---|
| KJ | 1,666 | 450 | — |
| Protein (N × 6.25) | 0.67 | 0.18 | — |
| Carbohydrates | 45.29 | 12.21 | — |
| Lipids | 2.38 | 0.64 | — |
| L-leucine | 13.889 | 3.8 | Essential |
| L-lysine | 7.222 | 2.0 | Essential |
| L-lsoleucine | 6.944 | 1.9 | Essential |
| L-valine | 6.944 | 1.9 | Essential |
| L-threanine | 3.889 | 1.1 | Essential |
| L-cysteine | 1.667 | 0.4 | Nonessential |
| L-histidine | 1.667 | 0.4 | Essential |
| L-phenyalanine | 1.111 | 0.3 | Essential |
| L-methionine | 0.556 | 0.2 | Essential |
| L-tyrosine | 0.333 | 0.1 | Nonessential |
| L-tryptophan | 0.222 | 0.1 | Essential |

Option ingredients added to formula:
D-phenyalanine - 1.11 g/day;
Chromium salt - 100-1000 micrograms per day;
Rhdiola rosea 100-500 g/day.

Cogenix

| | Actual | total | |
|---|---|---|---|
| Ingredient Elemental | g/serv | KG | Amount (%) |
| Vit D3 500 IU/mg | 100 | 0.004 | 0.12480 |
| Chrom Polynicotinate | 10 | 0.00 | 0.15600 |
| 5HTP | 95 | 0.105 | 0.00316 |
| D1-Phenyalalnine | 100 | 0.500 | 0.01500 |
| Rhodiola rosea ext 1% | 100 | 0.100 | 0.00300 |
| L-Glutamine | 100 | 0.025 | 0.00750 |
| L-Tyrosine | 100 | 0.350 | 0.10500 |
| Taurine | 100 | 0.200 | 0.00600 |
| L-Glutathione | 100 | 0.025 | 0.00075 |
| Gylcine-Propionyl Carnitine | 100 | 0.500 | 0.0.0150 |
| Acetyl-L-Carnitine Arginate | 100 | 0.500 | 0.00150 |
| Ginko Biloba leaf Ext 24% | 100 | 0.200 | 0.00600 |
| Ginseng root, Korean panax | 100 | 0.200 | 0.00600 |
| Zinc Chelate | 20 | 0.100 | 0.00300 |
| Pyridoxine HCL | 82 | 0.037 | 0.00110 |
| Pyridoxyl 5′ Phosphate | 61 | 0.049 | 0.00148 |
| Citric Acid | 100 | 0.200 | 0.00600 |
| Potassium Bicarbonate | 100 | 0.104 | 0.00312 |
| Phosphatidyl Choline | 40 | 1.375 | 0.04125 |
| Phosphatidyl Serine | 20 | 1.000 | 0.03000 |

Optional Ingredients:Ascorbic Acid 10-500 mg; Vitamin E—a antioxidant amount;

Huperazine A—1-3.5 mg; Ferulic acid 10-500 mg; GlyceroPhosphoCholine 400 mg/day

What is claimed is:

1. A composition comprising, in combination:
a. a pre-formed, pro-liposome delivery system selected from the group consisting of an oral tablet, a sublingual tablet, a caplet, a capsule, and an aerosol, wherein the pre-formed, pro-liposome delivery system comprises at least one liposome forming ingredient member and at least two chemically distinct disintegrating agents;
b. a biologically active homeopathic active ingredient dispersed in the pre-formed, pro-liposome delivery system;
wherein after ingestion of the composition the pre-formed, pro-liposome delivery system disintegrates in a patient's alimentary and at least a portion of the at least one liposome forming ingredient member encapsulates in in situ-formed liposomes at least a portion of the biologically active homeopathic active ingredient.

2. A composition according to claim 1 wherein the in situ-formed liposomes have a vesicle size distribution of about 10 nm to about 200 nm.

3. A composition according to claim 1 wherein the composition comprises a tablet that comprises about 77% excipients by weight, about 17% disintegrating agents by weight, about 1% of biologically active homeopathic active ingredient by weight, and about 5% phospholipids and phospholipid derivatives by weight.

4. A composition according to claim 1 that further comprises at least one preservative selected from the group consisting of ethanol, a benzoic acid salt, and a sorbic acid salt.

5. A composition according to claim 1 that further comprises a growth hormone and/or an allopathic ingredient.
